(19) United States
(12) Patent Application Publication
(10) Pub. No.: US 2012/0329885 A1
(43) Pub. Date: Dec. 27, 2012

(54) Ultraviolet light absorbing compounds based on benzyl substituted 2-(2- hydroxyphenyl) benzotriazoles

(76) Inventor: Chia-Hu Chang, Tappan, NY (US)

(21) Appl. No.: 13/135,020

(22) Filed: Jun. 23, 2011

(51) Int. Cl. A61K 8/49; A61Q 19/00; C09K 3/00; C07D 249/18; C08K 5/3475

(52) U.S. Cl. 514/788; 548/260; 524/91; 252/182.29

(57) ABSTRACT

The present invention relates to ultraviolet light absorbing compounds having the formula I and compositions containing these compounds in combination with polymers and/or carriers:

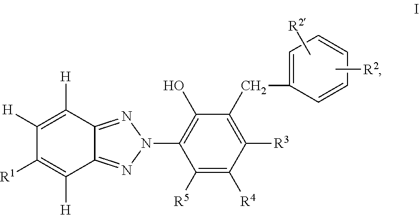

wherein;
   R¹ is hydrogen or a halogen atom or a straight-chain or branched alkyl group, an alkoxy group or a carboalkoxy group;
   R² and R^{2′} can be the same or different and individually are selected from the group consisting of hydrogen, an unsubstituted or substituted straight-chain or branched alkyl group, an alkoxy group, a carboxyalkyl group, a carboalkoxy, a carboxyl group, an hydroxyl group, and a halogen atom.
   R³, R⁴ and R⁵ can be the same or different and individually are selected from the group consisting of hydrogen, a straight-chain or branched alkyl group, an alkylaryl group, an alkoxy group, a phenyl or substituted phenyl group, a cycloalkyl group, a carboalkoxy group, an arylalkyl group, an hydroxyl group and a halogen atom.

### FIELD OF INVENTION

The invention relates to ultraviolet light absorbers useful as protective agents and stabilizers in coatings, plastics and topically applied products. More particularly, the invention concerns compounds which are benzyl derivatives of 2-(2-hydroxyphenyl)benzotriazole and to compositions containing these UV absorbing compounds.

### BACKGROUND OF INVENTION

A problem in the art of manufacturing and processing thermoplastic polymers and coating compositions is their instability upon extended exposure to ultraviolet (UV) light sources. It is well known that light, oxygen and heat cause degradation of polymers resulting in deterioration of mechanical and physical properties of the polymer. Coatings and plastics tend to demonstrate unwanted color changes and reduced mechanical strength upon exposure to UV radiation. To prevent or at least retard the damage caused by these factors, UV light absorbing compounds are added to the plastic as stabilizers.

Benzotriazoles have long been an important class of UV absorbers and have gained wide commercial importance and acceptance as UV stabilizers for many industrial applications. The prior art is replete with references to their manufacture and utility including: U.S. Pat. No. 3,004,896 to Heller et al; U.S. Pat. No. 4,275,004 to Winter et al.

To improve the compatibility of the benzotriazole and reduce its volatility, appropriate side chains have been added to the phenolic ring or the benzene ring of the benzotriazole.

Since the types of UV light sensitive polymers and compositions employed in different applications is diverse there is not a single set of side chain substituents or “modifying groups” that will provide an optimal set of properties for these diverse chemical systems. Furthermore, in most cases the modifying groups do not add substantially to the UV absorption characteristics of the molecule, such that the additional molecular weight of the modifying group does not contribute to the light absorption properties of the UV light stabilizer. Consequently, the UV absorption properties of the modified stabilizer can actually be reduced because of a relative lowering in molar extinction coefficient.

The current invention describes 2-(2-hydroxyphenyl)benzotriazole that are substituted at the 3 position (ortho to the hydroxyl group) of the hydroxyphenyl ring with a benzyl group or a substituted benzyl group (i.e., wherein one or more hydrogen atoms on the benzene ring is substituted while preserving the benzyl structure). To our knowledge these compounds heretofore have not been commercially available and have not been reported in the literature. It has been found that these 2-(2-hydroxy-3-benzyl phenyl)benzotriazole derivatives have a surprisingly higher molar UV absorption coefficient than 2-(2-hydroxyphenol)benzotriazoles that are substituted ortho and/or para to the hydroxyl group by a cumyl group (i.e., dimethyl benzyl) or a substituted cumyl group such as those described for example in U.S. Pat. No. 4,226,763 and U.S. Pat. No. 4,278,589 to Dexter et al.

### SUMMARY OF THE INVENTION

The invention provides ultraviolet light absorbing compounds having formula I:

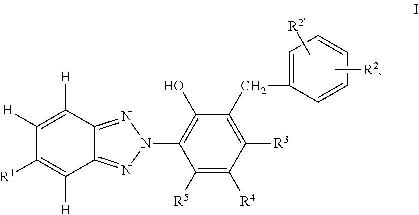

The compounds of the invention have two key structural features. Firstly, the two phenyl groups are separated by an unsubstituted methylene bridging group, i.e., a —CH₂— group. Secondly the compounds are asymmetric with respect to this methylene group, i.e. the methylene group separates two different chemical substituents.

R¹ is hydrogen or a halogen atom or a straight-chain alkyl group containing 1 to 8 carbon atoms or a branched alkyl group containing 3 to 8 carbon atoms or an alkoxy group containing 1 to 4 carbon atoms, or a carboalkoxy containing 2 to 9 carbon atoms;

R² and R^{2′} can be the same or different and individually are selected from the group consisting of hydrogen, an unsubstituted or substituted straight-chain alkyl group of 1-30 carbon atoms, an unsubstituted or substituted branched alkyl group of 3-15 carbon atoms, an alkoxy group of 1 to 12 carbon atoms, a carboxyalkyl group of 2 to 8 carbon atoms, a carboalkoxy group of 2 to 12 carbon atoms, a carboxyl group, an hydroxyl group, and a halogen atom.

Since R² and R^{2′} can be the same or different the designation of the benzyl substituent by the structural formula:

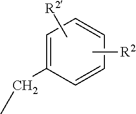

is meant to represent compounds falling within the scope of the invention that have either one or two substituent groups on the benzene ring regardless of their position relative to the methylene group, i.e., mono or di-substituted benzyl groups wherein the substituents are located on the benzene ring.

R³, R⁴ and R⁵ can be the same or different and individually are selected from the group consisting of hydrogen, a straight-chain alkyl containing 1 to 16 carbon atoms; a branched alkyl containing 3 to 12 carbon atoms; an alkylaryl group of 7 to 12 carbon atoms; an alkoxy group of 1 to 12 carbon atoms, a phenyl group, a phenyl group substituted with an alkyl group of 1 to 8 carbon atoms, a cycloalkyl group of 5 to 6 carbon atoms, a carboalkoxy group of 2 to 9 carbon atoms, an arylalkyl group of 7 to 9 carbon atoms, an hydroxyl group and a halogen atom.

The invention further encompasses compositions of matter which include the ultraviolet light absorbing compound having formula (I). One embodiment of such a composition is an ultraviolet light (UV) absorbing compositions that includes a carrier and from 0.01% to 10% of the UV light absorbing ketone of formula (I). These compositions function as a protective coating or film when applied to a substrate which is susceptible to UV damage.

In another embodiment, the composition of matter includes an organic material which is subject to UV light-induced deterioration, e.g., a plastic, in combination with from 0.1% to 10%, preferably 0.1% to 5% by weight of the UV light absorbing ketone of formula (I).

The invention also encompasses a process for the preparation of the UV light absorbing benzyl substituted 2-(2-hydroxyphenol)benzotriazoles compounds starting from the 2-(2-hydroxyphenol)benzotriazoles by reduction of a benzoyl substituted 2-(2-hydroxyphenol) benzotriazole.

These and other variations of the inventive compounds, process and compositions disclosed herein will become clear from the description of the invention which follows.

### BRIEF DESCRIPTION OF DRAWINGS

FIG. 1 is a graph comparing the UV absorption spectra of a compound of the invention, compound 1 (2-(2-hydroxy-3-benzyl-5-methyl-phenyl)benzotriazole (Curve A) with (2(2-hydroxy-3-benzoyl-5-methyl-phenyl)benzotriazole (Curve B) and a commercially available UV absorbers 2-(2-hydroxy-3-cumyl-5-tert. octyl-phenyl)benzotriazole (Curve C).

### DETAILED DESCRIPTION OF THE INVENTION

Unless otherwise indicated, % or wt % as used herein refers to percent by weight of an ingredient as compared to the total weight of the composition or component that is being discussed.

Except in the operating and comparative examples, or where otherwise explicitly indicated, all numbers in this description indicating amounts of material or conditions of reaction, physical properties of materials and/or use are to be understood as modified by the word “about.” All amounts are by weight of the final composition, unless otherwise specified.

For the avoidance of doubt the word “comprising” is intended to mean “including” but not necessarily “consisting of” or “composed of.” In other words, the listed steps or options need not be exhaustive.

The invention relates to ultraviolet light (hereinafter designated “UV”) absorbing compounds, their preparation and specific types of compositions which incorporate these compounds. The UV absorbing compounds and compositions in which they are incorporated are described in detail below including preferred embodiments and optional and alternative ingredients.

### UV Absorbing Compounds

The UV absorbing compounds of the invention are 2-(2-hydroxyphenyl)benzotriazoles which incorporate a benzyl substituent adjacent (designated as the “3” position) to the hydroxyl group (designated the “2” position) of the “central” phenyl group. The compounds have the formula (I):

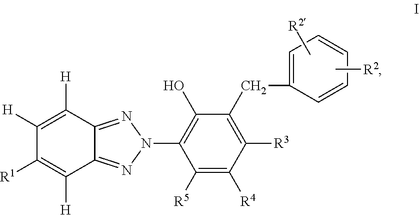

In the compounds of the invention the two differently substituted phenyl groups are separated by an unsubstituted methylene bridging group and are thus asymmetric with respect to this methylene group.

R¹ is a hydrogen or a halogen atom or a straight-chain alkyl group containing 1 to 8 carbon atoms or a branched alkyl group containing 3 to 8 carbon atoms or an alkoxy group containing 1 to 4 carbon atoms, or a carboalkoxy containing 2 to 9 carbon atoms;

R¹ can be an alkyl group of 1 to 8 carbon atoms such as methyl, ethyl or n-butyl.

R¹ can be a halide such as chlorine, R¹ can also be lower alkoxy of 1 to 4 carbon atoms such as methoxy, ethoxy or n-butoxy. R¹ can also be carboalkoxy containing 2 to 9 carbon atoms such as carbomethoxy, carboethoxy, or carbo-n-octoxy.

Preferably R¹ is hydrogen or a halide or a lower alkyl group containing 1 to 3 carbon atoms or a methoxy group.

Most preferably R¹ is hydrogen or chlorine.

R² and R^{2′} can be the same or different and is selected from the group consisting of hydrogen, an unsubstituted or substituted straight-chain alkyl group of 1-30 carbon atoms, an unsubstituted or substituted branched alkyl group of 3-15 carbon atoms, an alkoxy group of 1 to 12 carbon atoms, a carboxyalkyl group of 2 to 8 carbon atoms, a carboalkoxy group of 2 to 12 carbon atoms, a carboxyl group, an hydroxyl group, and a halogen atom.

R² and R^{2′} can be a hydrogen atom.

R² and R^{2′} can be a straight chain alkyl group having 1-22, preferably 1-18 and most preferably 1-9 carbon atoms such as a methyl, ethyl, propyl, or nonyl group.

R² and R^{2′} can be a branched alkyl group having 3-15, preferably 3-12 carbon atoms such as isopropyl, amyl, tert. butyl or tert octyl.

R² and R^{2′} can be an alkoxy group having 1 to 4 carbon atoms such as methoxy, ethoxy, n-butoxy.

R² and R^{2′} can be a carboxyalkyl group having 1 to 4 carbon atoms such as carboxymethyl, carboxyethyl, or carboxypropyl.

R² and R^{2′} can be a carboalkoxy group having 2 to 9 carbon atoms such carbomethoxy, carboethoxy, carbo-n-butoxy, or carbo-n-octoxy.

R² and R^{2′} can be carboxyl group. R² and R^{2′} can be a hydroxyl group.

R² and R^{2′} can be a halogen atom such as chlorine or fluorine.

R² and R^{2′} can be a straight-chain alkyl of 1 to 22 carbon atoms, or a branched alkyl of 3 to 18 carbon atoms.

R² and R^{2′} can be a straight-chain or branched alkyl substituted with an alkoxy group having 1-8 carbon atoms such as methoxy, ethoxy or tert. butoxy, or substituted with a carboxyl group or substituted an hydroxyl group or substituted with a phenyl group.

Preferably R² and R^{2′} is hydrogen or an alkyl group having 1 to 5 carbon atoms or an hydroxyl group or a carboxyl group.

R³, R⁴ and R⁵ can be the same or different and are selected from the group consisting of a hydrogen atom, a straight-chain alkyl containing 1 to 16 carbon atoms; a branched alkyl containing 3 to 12 carbon atoms; an alkylaryl group of 7 to 12 carbon atoms; an alkoxy group of 1 to 12 carbon atoms, a phenyl group, a phenyl group substituted with an alkyl group of 1 to 8 carbon atoms, a cycloalkyl group of 5 to 6 carbon atoms, a carboalkoxy group of 2 to 9 carbon atoms, an arylalkyl group of 7 to 9 carbon atoms, an hydroxyl group and a halogen atom.

R³, R⁴ and R⁵ can be alkyl group of 1 to 12 carbon atoms, preferably 1 to 8 carbon atoms such as methyl, ethyl, sec-butyl, tert-butyl, amyl or tert.-octyl.

R³, R⁴ and R⁵ can also be alkoxy of 1 to 5 carbon atoms such as methoxy, ethoxy or n-butoxy.

R³, R⁴ and R⁵ can also be phenyl substituted with alkyl groups, said alkyl groups having 1 to 5 carbon atoms such as methyl, tert-butyl, or tert-amyl.

R³, R⁴ and R⁵ can also be cycloalkyl of 5 to 6 carbon atoms such as cyclopentyl or cyclohexyl.

R³, R⁴ and R⁵ can also be carboalkoxy of 2 to 5 carbon atoms such as carbomethoxy, carboethoxy, carbo-n-butoxy.

R³, R⁴ and R⁵ can also be arylalkyl of 7 to 9 carbon atoms such as benzyl, α-methyl benzyl or α,α-dimethyl benzyl.

Preferably R³, R⁴ and R⁵ are selected from the group consisting of hydrogen, hydroxyl, methyl, tert-butyl, tert-amyl, sec-butyl, tert-octyl, cyclohexyl, carboxymethyl, carboxyethyl, methoxy, α-methylbenzyl and α,α-dimethylbenzyl (cumyl).

The convention used throughout in the naming of specific compounds falling with the scope of the invention is to treat them as derivatives of 2-(2-hydroxy phenyl)benzotriazoles wherein the benzyl group is a component of the substituent located at the 3 position on the central phenyl ring, i.e., ortho to the hydroxyl group on the central phenyl group (See Formula I).

Preferred variants of formula I have: R¹=hydrogen or chlorine; R² and R^{2′}=hydrogen or an alkyl containing 1 to 18 carbon or an alkyl group containing 1-18 carbon atoms substituted with and alkoxy group, a carboxyl group or an hydroxyl group; R³=hydrogen or hydroxyl; R⁴=methyl or ethyl or tert. butyl or amyl or cumyl or tert. octyl; and R⁵=hydrogen or hydroxyl. Preferably, at least one of the R² and R^{2′} substituents is hydrogen, and at least one on the R³, R⁴ and R⁵ is hydrogen.

The following are non-limiting examples of representative compounds of formula I: 2-[2-hydroxy-3-benzyl-5-methyl-phenyl]-benzotriazole, 2-[2-hydroxy-3-benzyl-5-isopropyl-phenyl]-benzotriazole, 2-[2-hydroxy-3-benzyl-5-tert.butyl-phenyl]-benzotriazole, 2-[2-hydroxy-3-benzyl-5-tert.octyll-phenyl]-5-chlorobenzotriazole, 2-[2-hydroxy-3-benzyl-5-cumyl-phenyl]-benzotriazole, 2-[2-hydroxy-3-benzyl-5-cumyl-phenyl]-5-chlorobenzotriazole, 2-[2-hydroxy-3-(3′-isopropyl benzyl)-5-tert.butyl-phenyl]-benzotriazole, 2-[2-hydroxy-3-(3′-methyl benzyl)-5-amyl-phenyl]-benzotriazole, 2-[2-hydroxy-3-(3′-methyoxy-benzyl)-5-tert. butyl-phenyl]-benzotriazole, 2-[2-hydroxy-3-(3′-methyl benzyl)-5-tert.butyl]-benzotriazole, 2-[2-hydroxy-3-(3′-methyoxy-benzyl)-5-methyl-phenyl]-benzotriazole.

Synthesis

The invention further provides a preferred processes for the preparation of the benzyl substituted benzotriazole of formula I by the thermal reduction of a benzoyl substituted 2-(2-hydroxyphenyl)-benzotriazoles, e.g.: 2-[2-hydroxy-3-benzoyl-5-methyl-phenyl]-benzotriazole, in the presence of a suitable catalyst.

The benzoyl substituted 2-(2-hydroxyphenyl)-benzotriazoles were prepared by reacting a 2-benzotriazolyl-4-substituted phenol with an acylating agent such as an acid halide, an acid or an anhydrides in the presence of a strong acid or Lewis acid or acylium ion salt to form an ester followed by a Fries rearrangement. The process is described in U.S. Pat. No. 7,874,103 B2 to Chia-Hu Chang issued Dec. 7, 2010 incorporated in its entirety by reference herein.

The process is illustrated in the following exemplary reaction scheme for the synthesis of 2-[2-hydroxy-3-benzyl-5-methyl-phenyl]-benzotriazole (Formula IV).

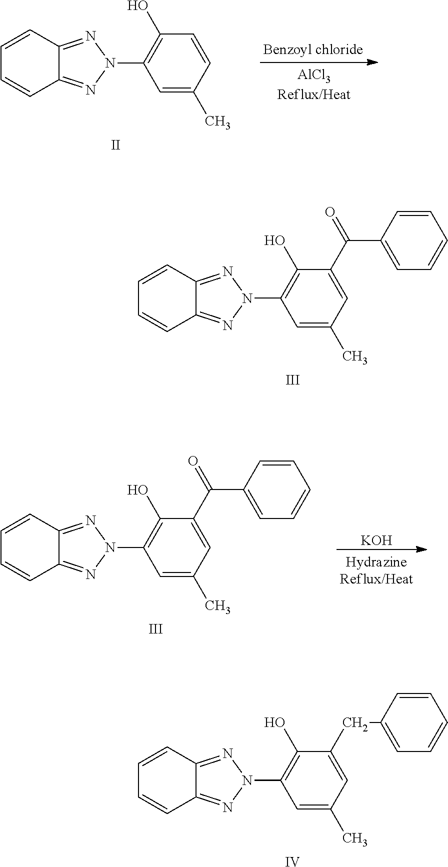

Here, 2-benzotriazolyl-4-methyl phenol (Formula II) is first reacted with benzoyl chloride to yield 2-[2-hydroxy-3-benzoyl-5-methyl-phenyl]-benzotriazole (Formula III). This synthesis actually proceeds in two stages one or both of which are carried out at a higher temperature.

The first stage is the reaction of the benzoyl chloride with the 2-hydroxy group of the benzotriazole to form an ester, in this case, 2-[2-carboxyphenyl-5-methyl-phenyl]-benzotriazole. This reaction can be carried out in an aprotic solvent such as methylene chloride, toluene, benzene, xylene, hydrocarbons such as hexane, heptane, petroleum ethers, and mineral spirits, cycloaliphatic ethers such as furan, tetrahydrofuran and dioxane.

The benzotriazole, acid chloride e.g., benzoyl chloride, acid catalyst (e.g., AlCl₃) and optional solvent are combined and heated under reflux conditions at atmospheric pressure. The ratio of benzotriazole and acid chloride is in the range from about 3:1 to about 1:3, preferable about 3:1 to about 1:1.5.

Suitable acid halides include acid chlorides. Suitable acid chlorides include unsubstituted or substituted benzoyl chloride. Suitable acids include benzoic acid or substituted benxoic acid. Suitable anhydrides include benzoic anhydride or substituted benzoic anhydride.

The hydroxyphenyl benzotriazole reactant may be prepared by any method known in the art including those taught in U.S. Pat. Nos. 5,097,041, 4,943,637 and 5,104,992. 2-aryl-2H-benzotriazoles monomers may be produced by reducing o-nitroazobenzenes through a 2-phenylbenzotriazole-N-oxide intermediate. A wide variety of reduction techniques is known. Reduction of o-nitroazobenzenes to 2-phenylbenzotriazole by zinc in the presence of sodium hydroxide is disclosed in U.S. Pat. Nos. 3,018,269; 3,230,194; 3,773,751; 4,041,044; and 4,224,451. Reduction using aldehyde reducing agents and aromatic ketone catalysts is disclosed in U.S. Pat. No. 4,835,284. Reduction using saccharides and an aromatic ketone catalyst is disclosed in U.S. Pat. No. 4,780,541. All of these patents are incorporated herein by reference. These show methods for the preparation of hydroxyphenyl benzotriazoles by reductive cyclization of azo dyes with saccharides in the presence of aromatic ketone catalysts, which act by receiving hydrogen from the reducing agent and giving hydrogen to a material to be reduced. In each of these cases, saccharide reduction is catalyzed by such aromatic ketone catalysts as substituted and unsubstituted fluorenone.

In the second stage, the solvent is removed by distillation and the temperature is increased (e.g., to 160° C.) to initiate a Fries rearrangement of ester which may have formed in step 1 to form the ketone through the migration of the acyl group (e.g., the benozyl group) to the ortho position relative to the hydroxy group since the para position is blocked by R³ which must be a substituent other than H since substitution at the para position is not desirable.

It should be noted that non-aromatic organic solvents with high boiling points are also suitable. If such a solvent is used there is no need to remove the solvent before Fries rearrangement is initiated. Suitable solvents include but not limited to dimethylsulfoxide, ethylene glycol monoacetate, ethylene glycol diacetate and diethylene glycol monobutyl ether).

The process conditions and intermediates of course depend upon the UV absorbing variant desired. Other compounds of the invention can be prepared in an analogous manner using for example, appropriately substituted 2-benzotriazolyl-4-hydrocarbyl phenol and appropriately substituted benzoyl chloride, substituted benzoic acid or substituted benzoic anhydride.

Once the Fries rearrangement is completed, The temperature of the reaction mixture formed in step 2 is first lowered to below about 90° C. and enough water is added until the cessation of bubbling. The benzoyl substituted 2-(2-hydroxyphenyl)benzotriazoles is then purified and isolated.

The benzoyl product (e.g., Formula III) can then be purified, for example, by successive extractions in which esters that may be present in the product mixture are hydrolyzed, converted to water soluble variants by hydrolysis and extracted into an aqueous phase. For example, aqueous HCl is first added to the organic phase and the mixture stirred under reflux conditions. The mixture is then cooled and the aqueous layer discharged. Aqueous NaOH is then introduced to the organic phase, the mixture again stirred at reflux conditions, cooled and the aqueous phase is again discharged. Another portion of aqueous HCl is then added to the organic phase, the mixture again refluxed under shear, cooled and the aqueous phase again discharged. Finally, the remaining organic phase is washed several times to obtain the Benzoyl derivative.

The second step of the process depicted in the above scheme is the reduction to benzoyl intermediate (Formula III) to 2-[2-hydroxy-3-benzyl-5-methyl-phenyl]-benzotriazole (Formula IV). The purified intermediate benzoyl derivative, e.g. 2-(2-hydroxy-3-benzoyl-5-methyl-phenyl)benzotriazole, is then dissolved in a polar solvent such as diethylene glycol monobutyl ether. To this mixture a strong base, e.g. NaOH (aqueous solution) and hydrazine are slowly added. The temperature is raised slowly until the solution boils. The solution is refluxed for an hour or more and the water is removed by distillation. When all the water has been removed, the temperature is raised to 250° C. and stirred for another hour and then cooled. During this process samples of the reaction mixture can be taken and analyzed for degree of conversion by GC or other appropriate analytical methods known in the art.

The product can be purified by precipitation with water, filtered and washed with water and optionally treated with an acidic aqueous solution and rinsed further with water. A quantitative illustration of the method is given in Example 1.

Several methods can be used to identify the intermediates, products and purity: i) UV spectroscopy to confirm that the UV spectrum contains the benzyl derivative which retains the characteristic double absorption peaks of 2-(2-hydroxy phenyl)benzotriazole at about 310 and 345 nm and has lost the benzophenone spectral features between 300 and about 250 nm which is characteristic of the benzoyl intermediate (i.e., before reduction); ii) proton NMR assignments to confirm the presence of the methylene group and the two types of phenyl protons in the case of the benzyl variant; and iii) gas chromatography which demonstrates a GC peak having a different retention time, compared to reactant 2-benzotriazolyl-4-methyl phenol.

For certain applications it is desirable to employ mixtures of the UV absorbing compounds having Formula I in which the individual components of the mixture differ in one or more of the R¹, R², R^{2′} or R³, R⁴ and R⁵ substituent groups. For example, when R² or R^{2′} is an alky group it is possible to reduce the tendency of the UV absorbing compound to crystallize from an organic solvent or polymer melt by utilizing a mixture of different UV compounds having different R² alkyl chain lengths, isomers or degree of branching. Such mixtures can display improved compatibility (e.g., solubility) especially in a polymer film or plastic.

To obtain mixtures of the UV absorbing compounds which differ in one or more R¹, R², R^{2′}, R³, R⁴ or R⁵ substituent groups, the different components of the mixture can of course be separately synthesized. However, in some cases it is much more convenient to form the mixture in situ by employing mixtures of different benzotriazole phenol and substituted benzoyl chloride reactants. The use of such mixed reactants in the synthesis described above to form mixtures of UV absorbing compounds having beneficial solubility; absorption or other physical or chemical properties is considered to be within the scope of the invention.

One advantageous mixture for certain applications is a mixture of the benzyl substituted compounds of formula I, e.g., 2-[2-hydroxy-3-benzyl-5-methyl-phenyl]-benzotriazole (Formula IV), with a benzoyl substituted 2-[2-hydroxy phenyl]-benzotriazole such as 2-[2-hydroxy-3-benzoyl-5-methyl-phenyl]-benzotriazole (e.g., formula III). The latter compounds are described in detail in U.S. Pat. No. 7,847,103 and for the current application can be regarded to have the general formula V

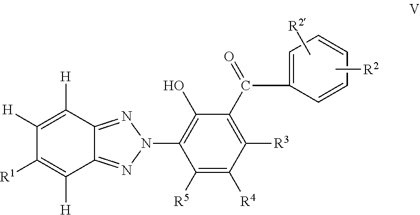

where R1, R2, R2′, R3, R4 and R5 have the same meaning as defined previously.

For purposes of the current invention, the compounds having formula I and formula V are designated as “benzyl substituted 2-[2-hydroxy phenyl]-benzotriazole]” and “benzoyl substituted 2-[2-hydroxy phenyl]-benzotriazole]” respectively.

Mixtures of benzyl and benzoyl substituted 2-[2-hydroxy phenyl]-benzotriazole] can be prepared by mixing individual synthesized compounds. However, it is again more convenient to prepare the mixture “in situ” by not carrying out the second step of the synthesis described above to completion, i.e., by only partially reducing the benzoyl derivative V to the benzyl derivative I. By carefully monitoring the GC profile and/or the UV spectra of the product a desired benzyl to benzoyl ratio can be achieved, for example in the range of 99.5:0.5 to 5:95, preferably 99:1 to 5:95, preferably 4:1 to 1:4, preferably 3:1 to 1:3.

Complex mixtures of the benzyl substituted 2-[2-hydroxy phenyl]-benzotriazole] of the invention with other types of ketones described in U.S. Pat. No. 7,847,103 can also be prepared in situ in which a small portion of the benzoyl chloride in step 1 above is replaced with a different acid chloride.

Applications

The compounds of formula I are broadly useful in protecting materials from damage caused by exposure to ultraviolet radiation. The compounds according to the present invention can either be incorporated directly into the materials to be protected from UV light rays, e.g., in a plastic, or they can be incorporated into protective coatings, foils and coverings. The protected material or protective covering can contain, for example 0.001% to 15% of the active UV absorbing ingredient, preferably 0.1 to about 10%, preferably 0.01 to 5%. In non-actinic agents for the human skin, the content of active agent should be advantageously 0.1% to 10% calculated on the non-volatile components of the preparation.

One embodiment of the invention is a composition of mater comprising an organic material subject to light-induced deterioration stabilized with from 0.1 to 5% by weight of one or more of the ultraviolet light absorbing compounds of the invention as described above. The UV absorbing compounds of the invention are especially suitable for protecting organic materials that are synthetic polymer such polyesters, polycarbonates, polysulfones, polyamides, acrylic resins, polyolefins, polystyrenes, polyurethanes and various other polymers and blends described in more detail below. However, the organic material can also be a natural polymer such as cellulose based polymers, e.g., wood. The types of materials are discussed in more detail below.

The UV absorbing compounds of the invention are especially suitable when the composition of matter is a thermoplastic or thermosetting polymer. Such compositions are generally formed under high shear at elevated temperatures. Because the UV absorbing compounds of the invention are thermally stable and not volatile they are not lost during processing of the composition.

Examples of ultraviolet light absorbing compound for use in the above compositions include but are not limited to: 2-[2-hydroxy-3-benzyl-5-methyl-phenyl]-benzotriazole, 2-[2-hydroxy-3-benzyl-5-isopropyl-phenyl]-benzotriazole, 2-[2-hydroxy-3-benzyl-5-tert.butyl-phenyl]benzotriazole, 2-[2-hydroxy-3-benzyl-5-tert. octyll-phenyl]-5-chlorobenzotriazole, 2-[2-hydroxy-3-benzyl-5-cumyl-phenyl]-benzotriazole, 2-[2-hydroxy-3-benzyl-5-cumyl-phenyl]-5-chlorobenzotriazole, 2-[2-hydroxy-3-(3′-isopropyl benzyl)-5-tert.butyl-phenyl]benzotriazole, 2-[2-hydroxy-3-(3′-methyl benzyl)-5-amyl-phenyl]-benzotriazole, 2-[2-hydroxy-3-(3′-methyoxy-benzyl)-5-tert. butyl-phenyl]-benzotriazole, 2-[2-hydroxy-3-(3′-methyl benzyl)-5-tert.butyl-phenyl]-benzotriazole, 2-[2-hydroxy-3-(3′-methyoxy-benzyl)-5-methyl-phenyl]-benzotriazole.

Another embodiment of the invention comprises an ultraviolet light absorbing composition including a carrier, and 0.01% to 10%, preferably 0.1% to 5% by weight of an ultraviolet light absorbing compound of formula I which is uniformly dispersed in the carrier.

The term “carrier” is used herein to broadly encompass any suitable material or combination of materials that comprise a protective coating, foil, film, or covering over a substrate which is susceptible to damage from UV light exposure. The carrier need not and often is not itself resistant to UV damage and thus the compounds of the invention may be used to protect the integrity and properties of the coating, film, foil or covering itself which may serve a range of useful purposes beyond UV protection.

Suitable carriers can include one or more polymers, preferably film forming polymers, such as polyesters, polyester resins, polyamides, vinyl polymers, cellulose ethers, cellulose esters, polyurethanes polyhydrocarbons, acrylic polymers, various latex polymers and alkyd resins, polypeptides such as gelatin, and various mixtures thereof.

Alternatively, the ultraviolet light absorbing composition can be used to protect living tissue from UV radiation. In this case the carrier is a cosmetically acceptable material, i.e., safe for topical application to living tissue, generally human tissue such as cosmetically acceptable ointments, creams, lotions, gels, sprays and the like. Such carriers include oils and waxes such as hydrocarbon oils and waxes, e.g., mineral oil, vegetable oils and waxes such as triglycerides and ester oils, silicone oils and waxes, such as volatile silicones and polydimenthylsiloxanes, fluorocarbon oils, water (generally containing a compatibilizing agent) and mixtures thereof.

The compounds of this invention are effective stabilizers in combination with a wide range of organic polymers which are susceptible to damage from UV radiation. Polymers and their mixtures which can be stabilized include: mono- or diolefins; polystyrene and it various copolymers; graft copolymers of styrene; halogen-containing vinyl polymers; acrylic polymers; copolymer of acrylic acid and one or more of its derivatives, and a melamine-formaldehyde resin; vinyl and allyl containing polymers and copolymers; homopolymers and copolymers derived from epoxides; polyacetals; polyalkylene oxides; polyurethanes and polyureas; polycarbonates; polysulfones; polyamides and copolyamides; polyesters; alkyd resins; unsaturated polyesters resins which are derived from copolyesters of saturated and unsaturated dicarboxylic acids with polyhydric alcohols as well as from vinyl compounds as cross-linking agents and also the halogen-containing, flame-resistant modifications thereof; natural polymers, for example cellulose, rubber, as well as the chemically modified homologous derivatives thereof, for example cellulose acetates, cellulose propionates and cellulose butyrates and the cellulose ethers, for example methyl cellulose. Other examples of polymers that could be stabilized by the UV absorbing compounds of the invention are discussed in U.S. Pat. No. 7,874,103 B2.

The compounds of this invention are effective stabilizers for a variety of organic substrates subject to light induced deterioration and are expected to be resistance to loss from a stabilized composition during high temperature.

In general, the UV stabilizers of this invention are employed at from about 0.1 to about 5% by weight of the stabilized composition, although this will vary with the particular substrate and application although a higher level can also be used. An advantageous range is from about 0.5 to about 3%.

The UV absorbing compounds (also designated as “light stabilizers” or simply “stabilizer”) of Formula I may readily be incorporated into the organic polymers by conventional techniques, at any convenient stage prior to the manufacture of shaped articles or coatings there from. For example, the stabilizer may be mixed with the polymer in dry powder form, or a suspension or emulsion of the stabilizer may be mixed with a solution, suspension, or emulsion of the polymer.

Fabricated polymer compositions, e.g., plastic parts or objects may be formed by heating the polymer to its softening point in an extruder and adding the UV absorbing compound and other additives to the melt to form a substantially uniform physical mixture.

Thermoplastic polymer materials susceptible to UV damage and the compounds of the invention can be blended in the desired quantities and heated to a temperature above the softening point of the polymer. The heating in an extruder and blending of the coupled compound are done as known in the art. The heating and blending can be done in either order, however, in the preferred embodiment, these processes are conducted simultaneously. The mixing may be conducted in any suitable equipment including a Banbury mixer, single or twin screw extruder, ribbon blender, injection molding machine, two-roll mill or the like, thus forming a substantially uniform blend of the polymer material and the coupled compound. The mixing step is usually conducted for from about 0.1 minutes to about 20 minutes, preferably to about 10 minutes, at a temperature ranging from about 38° C. to about 350° C.

The UV absorbing materials of the invention are useful in stabilizing thermosetting and thermoplastic acrylic resins, thermocrosslinkable acrylic, alkyd or polyester resins which are used for automobile finishes and similar coating applications. These substances are described in “Encyclopedia of Polymer Science and Technology”, Interscience Publishers, New York, Volume 1 (1964), on pages 273 to 276 and Volume 13 (1970), on pages 530 to 532, and also in “Understanding Paint” by W. R. Fuller in American Paint Journal, St. Louis, 1965, pages 124 to 135.

Acrylic, alkyd or polyester resins and resin lacquers which can be stabilized in accordance with the invention against the action of light, oxygen and moisture are the customary stoving lacquers, for example those described in H. Kittel's “Lehrbuch der Lacke and Beschichtungen” (“Textbook of Lacquers and Coatings”), Volume 1, Part 2, pages 735 and 742 (Berlin, 1972), and in H. Wagner, H. F. Sarx, “Lackkunstharze” (“Synthetic Resins for Lacquers”), pages 229 to 235.

The stabilization, by means of the compounds according to the invention, of metal effect lacquers based on an acrylic resin which can be crosslinked by heat is another relevant application. In order to achieve the metal effect, the conventional aluminum pigments are used in a concentration of 1 to 10% by weight, relative to solvent-free binder (lacquer resin). The stabilized lacquers can be applied by the conventional one-coat or two-coat processes. In the latter case the initial lacquer containing the pigment is first applied and is then overlaid with transparent lacquer. The lacquer preferably contains 0.5 to 5% of the UV absorbing compounds of the invention.

Further additives which can be present in the lacquer are other customary light stabilizers, phenolic antioxidants, pigments, dyes, metal deactivators and the like.

The UV absorbing compounds are also useful in stabilizing stoving lacquers especially two-layer uni-lacquer coatings based on thermocrosslinkable acrylic, alkyd or polyester resins, which if desired can be crosslinked with melamine/formaldehyde resins, epoxide resins or polyisocyanates against the action of light.

The UV absorbing compounds and any additional additives used for stabilization according to the invention can be incorporated either only into the unpigmented topcoat lacquer or both into the unpigmented topcoat lacquer and into the undercoat lacquer containing pigment. Preferably, the light stabilizer is incorporated in the unpigmented topcoat lacquer such as topcoat lacquers based on acrylic/melamine resins.

The invention also relates further to stabilized organic material which is in the form of photographic material or is part of a photographic material, the photographic material containing, preferably in top layers, 0.1 to 5% by weight, relative to the photographic material without stabilizer, of the UV absorbing compounds according to the invention.

Although the compounds of the invention may be used above to provide a light stabilizing function, the compounds of this invention are often combined with other stabilizers, even other light stabilizers, in the preparation of stabilized compositions. The stabilizers may be used with phenolic antioxidants, pigments, colorants or dyes, light stabilizers such as hindered amines, metal deactivators, etc.

Examples of suitable materials that can be incorporated as optional ingredients, typically at levels from about 0.1 to about 10%, preferably from about 0.5 to about 5% by weight include: antioxidants; and light stabilizers such as esters of optionally substituted benzoic acids; sterically hindered amines or amino ethers such as are described in U.S. Pat. No. 4,547,537 to Ackerman et al incorporated by reference herein, sterically hindered amines such as those based on 2,2,6,6-tetraalkylpiperidine as described is U.S. Pat. No. 4,314,933 to Berner et al incorporated by reference herein, so-called N—OR₁-substituted hindered amines disclosed in U.S. Pat. No. 5,112,890 to Behrens et al incorporated by reference herein.

Other additives that can be incorporated in the stabilized compositions are thio-synergists such as dilauryl thiodipropionate, lubricants such as stearyl alcohol, fillers and reinforcing agents, asbestos, kaolin, talc, glass fibers, pigments, optical brighteners, flameproofing agents and antistatic agents, blowing agents, calcium carbonates, silicates, barium sulfate, metal oxides and hydroxides, carbon black and graphite.

Sterically hindered amine light stabilizers are especially useful in combination with the UV absorbing compounds of the invention to provide additional protection in coatings especially against light, water and humidity. The combination of sterically hindered amines and the stabilizers according to the invention makes possible excellent retention of gloss when exposed to weathering as well as resistance to blistering and flaking in stoving lacquer coatings such as are used for application of automobile top lacquers and acid catalyzed thermoset resin coating compositions to improve resistance against the deleterious effects of light, moisture and oxygen without inhibition of curing time.

The light stabilizing compounds of the invention can also be used in combination with other classes of UV absorbing compounds such as benzophenones, benzotriazoles, acrylic acid derivative, aryl-s-triazines, organic nickel compounds and oxanilides.

As discussed above, one advantageous mixture in certain applications is a mixture of the benzyl substituted-[2-hydroxy phenyl]-benzotriazole compounds of formula I with one or more ketones of 2-[2-hydroxy phenyl]-benzotriazole described in U.S. Pat. No. 7,847,103.

A preferred embodiment of the invention is an ultraviolet light absorbing composition that includes one or more carriers and/or one or more polymers that have been discussed above in combination with a UV absorbing compound which is a mixture of one or more benzyl substituted 2-[2-hydroxy phenyl]benzotriazole having formula I and one or more benzoyl substituted 2-[2-hydroxy phenyl]benzotriazole having the formula V. In other words the mixture of carrier and/or polymer and benzyl substituted 2-[2-hydroxy phenyl]benzotriazole further includes benzoyl substituted 2-[2-hydroxy phenyl]benzotriazole.

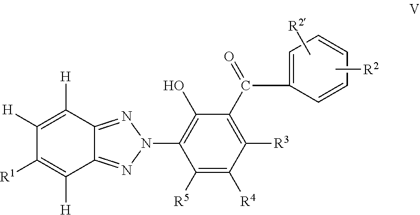

wherein the substituents R¹, R², R^{2′}, R³, R⁴ and R⁵ have the same meanings defined above.

The ratio of weight of the benzyl substituted 2-[2-hydroxy phenyl]benzotriazole to the weight of the benzoyl substituted 2-[2-hydroxy phenyl]benzotriazole can be in the range of 99.5:0.5 to 5:95, preferably 4:1 to 1:4 and preferably 3:1 to 1:3.

Preferably the substituents R¹, R², R^{2′}, R³, R⁴ and R⁵ of the benzyl substituted 2-[2-hydroxy phenyl]benzotriazole are the same as the substituents R¹, R², R^{2′}, R³, R⁴ and R⁵ of the benozyl substituted 2-[2-hydroxy phenyl]benzotriazole. That is to say R¹ of the benzoyl substituted 2-[2-hydroxy phenyl]benzotriazole is the same as R¹ of benzyl substituted 2-[2-hydroxy phenyl]benzotriazole, e.g, a hydrogen atom, and so forth. This condition of equality of substitutents can be achieved by following the synthesis process described above where the benzoyl substituted 2-[2-hydroxy phenyl]benzotriazole of formula V is partially reduced to achieve a desired weight ratio of the benzyl substituted 2-[2-hydroxy phenyl]benzotriazole to benzoyl substituted 2-[2-hydroxy phenyl]benzotriazole.

### EXAMPLES

The following examples are shown as illustrations of the invention and are not intended in any way to limit its scope.

### Example 1

### Preparation of 2-(2-Hydroxy-3-Benzyl-5-Methyl-Phenyl)Benzotriazole

164.5 gm of 2-(2-hydroxy-3-benzoyl-5-methyl-phenyl)benzotriazole was dissolved in 400 gm diethylene glycol monobutyl ether and 80 gm 45% NaOH and 60 gm of 40% hydrazine were added slowly into the solution. The temperature was raised slowly until the solution boiled after which it was allowed to reflux for another hour. The water was then removed via distillation until no more water was obtained. The temperature was then raised to 250° C. and mixture was stirred for another hour. The solution was then cooled to 90° C. before 500 gm water was added. The temperature was then raised and the solution was allowed to boil for about 30 minutes. The solution was then cooled to the room temperature, filtered and washed with water several times. Gray flake-like solid material precipitated from the solution over night. The flake-like solid was placed into 500 gm water and 50 gm concentrated hydrochloric acid was added and the mixture was then allowed to reflux for one hour before the solution was cooled down to room temperature. Gray color powder which precipitated from the solution was then washed with water several times.

The product designated compound 1 had a melting point of 140-143° C. The yield was about 68%.

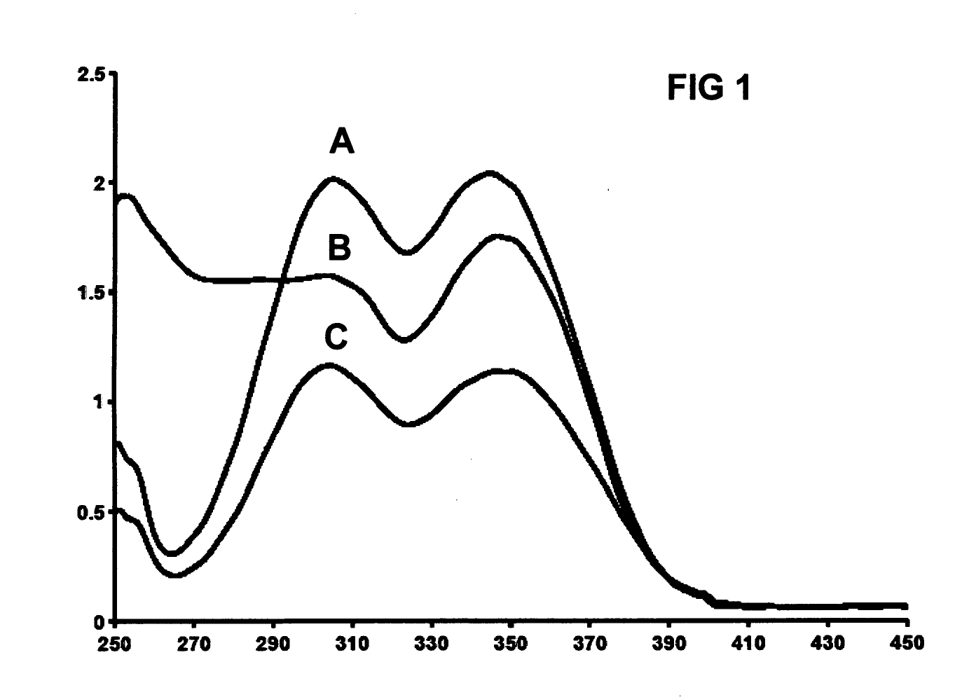

Confirmation that the product was 2-(2-hydroxy-3-benzyl-5-methyl-phenyl)benzotriazole was provided by a comparison of the UV spectra given in FIG. 1. Curve A is the spectrum of the product, compound 1 (2-(2-hydroxy-3-benzyl-5-methyl-phenyl)benzotriazole). Curve B is the spectrum of the starting material, 2-(2-hydroxy-3-benzoyl-5-methylphenyl)benzotriazole. Curve C is the spectrum of 2-(2-hydroxy-3-cumyl-5-tert. Octylphenyl)benzotriazole a typical commercial benzotriazole UV absorber of the prior art.

Comparing curves A, B and C it is observed that compound 1 (curve A) retains the characteristic spectral features of the typical benzotriazole (curve C) present at about 310 and 350 nm although compound 1 has a much higher maximum absorption. In contrast, the spectrum of compound 1 does not contain the strong absorption at wavelengths below 300 nm characteristic of the benzophenone group in 2-(2-hydroxy-3-benzoyl-5-methylphenyl)benzotriazole (curve B). These spectral features are consistent with reduction of the benzoyl group to a benzyl group.

### Example 2

### Preparation of 2-(2-hydroxy-3-benzyl-5-tert. butyl-phenyl)benzotriazole

2-(2-hydroxy-3-benzyl-5-tert.-butyl-phenyl)benzotriazole would be anticipated to be made by the following process. whose structure would be confirmed by the methods set forth in Example 1.

185.5 gm of 2-(2-hydroxy-3-benzoyl-5-methyl-phenyl)benzotriazole is dissolved in 400 gm diethylene glycol monobutyl ether and 80 gm 45% NaOH and 70 gm of 40% hydrazine is added slowly into the solution. The temperature is raised slowly until the solution boils after which it is allowed to reflux for another hour. The water is then removed via distillation until no more water was obtained. The temperature is then raised to 250° C. and mixture was stirred for another hour. The solution is then cooled to 90° C. before 500 gm water is added. The temperature is then raised and the solution is allowed to boil for about 30 minutes. The solution is then cooled to the room temperature, filtered and washed with water several times. The product is precipitated from the solution and purified according to the method described in Example 1.

### Example 3

### Preparation of 2-(2-hydroxy-3-(3-methyl benzyl)-5-methyl-phenyl)benzotriazole

2-(2-hydroxy-3-(m-methyl benzyl)-5-methyl-phenyl)benzotriazole would be anticipated to be made by the reduction of 2-(2-hydroxy-3-(3-methyl benzoyl)-5-methylphenyl)benzotriazole following the methods employed in Example 1.

### Example 4

Benzyl Preparation of Mixture of 2-(2-Hydroxy-3-Benzyl-5-Methyl-Phenyl)Benzotriazole and 2-(2-Hydroxy-3-Benzoyl-5-Methyl-Phenyl)Benzotriazole

As discussed in the section on synthesis, mixtures of benzoly and benzyl substituted 2-(2-Hydroxy-Phenyl)Benzotriazole can be prepared in-situ by carrying out the reduction to only a partial extent. This process could be carried out as follows.

164.5 gm of 2-(2-hydroxy-3-benzoyl-5-methyl-phenyl)benzotriazole is dissolved in 400 gm diethylene glycol monobutyl ether and 80 gm 45% NaOH and 60 gm of 40% hydrazine is added slowly into the solution. The temperature is raised slowly until the solution boiled after which it was allowed to reflux. Water is removed via distillation. Samples are taken periodically during reflux/distillation and analyzed by GC or other analytical means well known in the art to assess degree of reduction, i.e., the degree of conversion of 2-(2-Hydroxy-3-Benzoyl-5-Methyl-Phenyl)Benzotriazole and 2-(2-Hydroxy-3-Benzyl-5-Methyl-Phenyl)Benzotriazole.

When the desired degree of conversion is achieved, e.g., 4:1 benzyl to benzoyl, the solution is cooled to 90° C. before 500 μm water is added. The temperature is then raised and the solution is allowed to boil for about 30 minutes. The solution is then cooled to the room temperature, filtered and washed with water several times. The product is then isolated and purified as desired.

### Examples 5

Extinction coefficient of 2-(2-hydroxy-3-benzyl-5-methyl-phenyl)benzotriazole compared with benzotriazoles of the prior art.

0.2185 gm compound 1 (2-(2-hydroxy-3-benzyl-5-methyl-phenyl)benzotriazole) was dissolved in 100 ml CHCl₃ to prepare a stock solution. 1.5 ml of stock solution was diluted in CHCl₃ to 100 ml to a final concentration of 2.19×10⁻⁵ gram/ml and an alequate was transferred to a 1 cm path-length quartz cuvette. A UV spectrum was acquired and the absorption peak(s) were determined.

The UV spectra of a commercially available UV absorbing benzotriazole derivative, TINUVIN 928 (2-(2-hydroxy-3-cumyl-5-tert. octyl-phenyl)benzotriazole) and 2-(2-hydroxy-3-benzoyl-5-methyl-phenyl)benzotriazole (the starting ketone) in CHCl₃ was obtained in an analogous manner.

Extinction coefficients of each of the above compounds at their absorption maxima were derived from Bear's Law, (i.e., Absorbance equals extinction coefficient times path-length times concentration (wt % or molarity). The results are collected in Table 1 below and indicate that the molar extinction coefficient of the UV absorbing compound of the invention (compound 1) is surprisingly about 25% higher than the dimethyl benzyl substituted benzotriazole of the prior art and about 13% higher than the starting ketone. Comparison of extinction coefficient by weight, indicates that compound 1 is much more efficient on a weight basis than either of the prior art UV absorbers.

TABLE 1
Compositions and properties of

| | | | Relative |
|---|---|---|---|
| | Melting | Relative Molar | Extinction |
| | Point | Extinction | Coefficient |
| Sample | (° C.) | Coefficient | by Weight |
| Tinuvin 928 @ 350 nm | 109-111 | 1 | 1 |
| Compound 1 @ 345 nm | 140-143 | 1.27 | 1.77 |
| 2-(2-hydroxy-3- | 166-167.8 | 1.13 | 1.52 |
| benzoyl-5-methyl- | | | |
| phenyl) benzotri- | | | |
| azole | | | |

### Examples 6

### Influence of Benzotriazole Stabilizers on Retention of Gloss Values of Thermoset Acrylic coatings after Accelerated Weathering

The influence on gloss retention of thermoset acrylic coatings of compounds I-3 as prepared in the above examples can be demonstrated by the following procedure. Several thermoset acrylic resins and an alkyd/acrylic resin systems can be formulated with 2% by weight of light absorber stabilizers and cast onto glass plates as 1μ thick coatings. The coatings can be cured by heating at elevated temperatures for selected periods of time, e.g., 120° C. to 150° C. for 20 to 30 minutes. The loss of benzotriazole light stabilizer during coating/curing can be ascertained by UV-absorption analysis of the coatings. Any decrease in absorbance of the coatings can be correlated to loss of benzotriazole stabilizer during the curing step.

These cured coatings can then be subjected to the accelerated weathering test involving alternating 4-hour periods of UV irradiation at 60° C. with a 4-hour period of condensation (rain) at 50° C. for each cycle for a total of 670 hours. Again any decrease in absorbance of the weathered coatings can be correlated to loss of the benzotriazole stabilizer during the curing and weathering period.

Formulations containing compounds I-3 are expected to consistently provide higher retention of gloss values after accelerated weathering relative to a control have no light absorbers.

### Example 7

### Influence of Benzotriazole Stabilizers on Retention of Gloss Values of Acrylic Enamels and Lacquers after Accelerated Weathering

The influence of the compound 1 as prepared in the above example on gloss retention can be demonstrated by the following procedure. Samples of several thermoset acrylic enamels and a thermoplastic acrylic lacquer can be formulated to include the UV light stabilizer.

Gloss values can be compared for the initial enamel or lacquer and after exposure to a multi-cycle accelerated weathering test where each cycle includes 4 hours of UV exposure at 60° C. and 4 hours of condensation (moisture) at 50° C.

Formulations containing compound 1 at a level of incorporation in the test pieces of 1% and 2% by weight are expected to give consistently higher gloss values after 200 cycles (800 hours) and 50 cycles (200 hours) of accelerated weathering relative to a control have no light absorbers.

### Example 8

### Stabilization of Polyethylene Terephthalate

The stabilization of polyethylene terephthalate can be demonstrated by the following procedure. 0.5% of the compound 1 of Example 1 can be added as a stabilizer to molten polyethylene terephthalate at 270° C. with stirring under a nitrogen atmosphere. The resulting formulated polymer can then be ground with solid carbon dioxide.

The stabilized composition can then be extruded at elevated temperature into a film with little loss of stabilizer expected. The film is then exposed to actinic radiation. The stabilized film is expected to retain desirable physical properties for a longer period than does a film prepared from unstabilized polyester.

### Example 9

The following example illustrates how the improvement in color protection of pigmented compositions provided by the UV absorbing compounds of the invention can be determined.

The UV absorbing compound 1 is dry blended with polyacetal pellets (DELRIN® 500P NC010, DuPont) using a mixer. The dry blend is extruded and pelletized using a twin screw extruder at a melt temperature of about 210° C. The pellets are molded into test plaques using an injection molder operated at about 210° C. The plaques are exposed in a Xenon-arc Weather-Ometer according to automotive test procedure SAE J1885. Exposure is measured in terms of the total irradiation, measured in kilojoules per square meter (kJ/m²). Color change in the exposed samples is determined by measuring the color of the exposed samples compared to the unexposed samples as color difference (ΔE) according to ASTM D2244.

Incorporation of compound 1 is anticipated to significantly reduce the change in color, i.e., color difference between the exposed and non-exposed samples relative to a control which does not contain the UV absorbing compounds.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

1. Benzyl substituted 2-[2-hydroxy phenyl]benzotriazole ultraviolet light absorbing compound having the formula I: 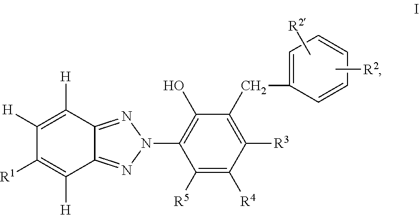
wherein;
R¹ is selected from the group consisting of hydrogen, a halogen atom, a straight-chain alkyl group containing 1 to 8 carbon atoms, a branched alkyl group containing 3 to 8 carbon atoms, an alkoxy group containing 1 to 4 carbon atoms and a carboalkoxy group containing 2 to 9 carbon atoms;
R² and R^{2′} can be the same or different and individually are selected from the group consisting of hydrogen, an unsubstituted or substituted straight-chain alkyl group containing 1-30 carbon atoms, an unsubstituted or substituted branched alkyl group containing 3-15 carbon atoms, an alkoxy group containing 1 to 12 carbon atoms, a carboxyalkyl group containing 2 to 8 carbon atoms, a carboalkoxy group containing 2 to 12 carbon atoms, a carboxyl group, an hydroxyl group, and a halogen atom; and
R³, R⁴ and R⁵ can be the same or different and individually are selected from the group consisting of a hydrogen atom, a straight-chain alkyl containing 1 to 16 carbon atoms; a branched alkyl containing 3 to 12 carbon atoms; an alkylaryl group containing 7 to 12 carbon atoms; an alkoxy group containing 1 to 12 carbon atoms, a phenyl group, a phenyl group substituted with an alkyl group containing 1 to 8 carbon atoms, a cycloalkyl group containing 5 to 6 carbon atoms, a carboalkoxy group containing 2 to 9 carbon atoms, an arylalkyl group containing 7 to 9 carbon atoms, an hydroxyl group and a halogen atom.

2. The ultraviolet light absorbing compound according to claim 1 wherein R¹ is selected from the group consisting of H, Cl, methyl, ethyl, tert. butyl, amyl and cumyl; and R² and R^{2′} is selected from the group consisting of hydrogen, a straight-chain or branched alkyl group containing up to 20 carbon atoms, an hydroxyl group, a halogen atom and an alkoxy group containing 1 to 8 carbon atoms; R³ and R⁵ are selected from the group consisting of hydrogen, hydroxyl and a straight or branched alkyl containing up to 8 carbon atoms; and R⁴ is selected from the group consisting of methyl, ethyl, tert. butyl, amyl, cumyl, tert. octyl;

3. The ultraviolet light absorbing compound according to claim 1 wherein R¹=hydrogen or chlorine; R² and R^{2′}=hydrogen or an alkyl containing 1 to 18 carbon or an alkyl group containing 1-18 carbon atoms substituted with an alkoxy group, a carboxyl group or an hydroxyl group; R³=hydrogen or hydroxyl; R⁴=is selected from the group consisting of methyl, ethyl, tert. butyl, amyl, cumyl, and tert. octyl; and R⁵=hydrogen or hydroxyl.

4. The ultraviolet light absorbing compound according to claim 3 wherein at least one of R² and R^{2′} is hydrogen and wherein R³ and R⁵ are both hydrogen.

5. The ultraviolet light absorbing composition comprising:
i) a carrier, and
ii) 0.01% to 10% of a benzyl substituted 2-[2-hydroxy phenyl]benzotriazole ultraviolet light absorbing compound according to claim 1 which is uniformly dispersed in said carrier.

6. An ultraviolet light absorbing composition according to claim 5, wherein the carrier comprises a polymer selected from the group consisting of polyesters, polyester resins, polyamides, vinyl polymers, cellulose ethers, cellulose esters, polyurethanes polyhydrocarbons, acrylic polymers, latex polymers, polypeptides, alkyd resins and mixtures thereof.

7. The ultraviolet light absorbing composition according to claim 5 wherein the polymer is film forming.

8. The ultraviolet light absorbing composition according to claim 5 further comprising a benzoyl substituted 2-[2-hydroxy phenyl]benzotriazole having the formula V 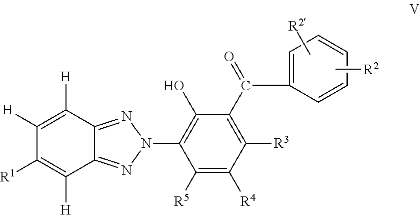
wherein the substituents R¹, R², R^{2′}, R³, R⁴ and R⁵ have the same meanings recited in claim 1.

9. The ultraviolet light absorbing composition according to claim 8 wherein the ratio of weight of the benzyl substituted 2-[2-hydroxy phenyl]benzotriazole to the weight of the benzoyl substituted 2-[2-hydroxy phenyl]benzotriazole is in the range of 99.5:0.5 to 5:95 and wherein the substituents R¹, R², R^{2′}, R³, R⁴ and R⁵ of the benzyl substituted 2-[2-hydroxy phenyl]benzotriazole are the same as the substituents R¹, R², R^{2′}, R³, R⁴ and R⁵ of the benozyl substituted 2-[2-hydroxy phenyl]benzotriazole.

10. The ultraviolet light absorbing composition according to claim 9 wherein the benzyl substituted 2-[2-hydroxy phenyl]benzotriazole is formed by partially reducing the benzoyl substituted 2-[2-hydroxy phenyl]benzotriazole of formula V to achieve a weight ratio of the benzyl to benzoyl substituted 2-[2-hydroxy phenyl]benzotriazole of from 99:1 to 5:95

11. The ultraviolet light absorbing composition according to claim 5 wherein the composition further comprises a dye or a pigment, or metal particles or a combination thereof.

12. The ultraviolet light absorbing composition according to claim 5, wherein the carrier comprises a cosmetic composition selected from the group consisting of ointments, creams, lotions, gels and sprays.

13. The ultraviolet light absorbing composition according to claim 5 further comprising at least one sterically hindered amine stabilizer selected from the group consisting of polyalkylpiperidines, N—OR substituted hindered amines and combinations thereof.

14. A composition of matter comprising from 0.1 to 10% by weight of a benzyl substituted 2-[2-hydroxy phenyl]benzotriazole ultraviolet light absorbing component according to claim 1 and a polymer selected from the group consisting of polyesters, polycarbonates, polysulfones, polyamides, acrylic resins, polyolefins, polystyrenes polyvinylchloride, polyureas, polyurethanes, polypeptides, cellulose polymers and combinations thereof.

15. The composition of matter according to claim 14 wherein the synthetic or natural polymer is a thermoset or thermoplastic polymer.

16. The composition of matter according to claim 14 further comprising at least one sterically hindered amine stabilizer selected from the group consisting of polyalkylpiperidines, N—OR substituted hindered amines and combinations thereof.

17. The composition of matter according to claim 14 further comprising a benzoyl substituted 2-[2-hydroxy phenyl]benzotriazole having the formula V 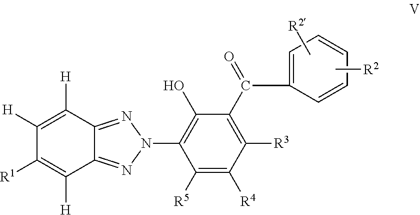
wherein the substituents R¹, R², R^{2′}, R³, R⁴ and R⁵ have the same meanings recited in claim 1.

18. The composition of matter according to claim 17 wherein the ratio of weight of the benzyl substituted 2-[2-hydroxy phenyl]benzotriazole to the weight of the benzoyl substituted 2-[2-hydroxy phenyl]benzotriazole is in the range of 99.5:0.5 to 5 to 95 and the substituents R¹, R², R^{2′}, R³, R⁴ and R⁵ of the benzyl substituted 2-[2-hydroxy phenyl]benzotriazole are the same as the substituents R¹, R², R^{2′}, R³, R⁴ and R⁵ of the benozyl substituted 2-[2-hydroxy phenyl]benzotriazole.

19. The composition of matter according to claim 18 wherein the benzyl substituted 2-[2-hydroxy phenyl]benzotriazole is formed by partially reducing the benzoyl substituted 2-[2-hydroxy phenyl]benzotriazole of formula V to achieve a weight ratio of the benzyl to benzoyl substituted 2-[2-hydroxy phenyl]benzotriazole of from 99:1 to 5:95.
